(19) United States
(12) Patent Application Publication
(10) Pub. No.: US 2022/0280108 A1
(43) Pub. Date: Sep. 8, 2022

(54) SYSTEMS AND METHODS FOR CALCULATING AN IMPACT SCORE

(71) Applicant: Force Impact Technologies, Inc., Gilbert, AZ (US)

(72) Inventors: Anthony M. Gonzales, Canoga Park, CA (US); Robert M. Merriman, Gilbert, AZ (US); Susan M. Merriman, Gilbert, AZ (US); Christopher T. Cooper, Stittsville (CA); Victoria A. Smith, Tempe, AZ (US); August G. Domel, Sleepy Hollow, IL (US)

(21) Appl. No.: 17/687,627

(22) Filed: Mar. 5, 2022

Related U.S. Application Data
(60) Provisional application No. 63/157,418, filed on Mar. 5, 2021.

(51) Int. Cl. A61B 5/00

(52) U.S. Cl. CPC A61B 5/682; A61B 5/7282; A61B 5/7275; A61B 2503/10; A61B 2562/0219

(57) ABSTRACT

A method for characterizing impacts experienced by a user comprises receiving a plurality of types of data, determining an impact score for the user, and communicating the impact score to the user. The plurality of types of data includes impact data associated with one or more impacts experienced by the user, and demographic data associated with the user. The impact score characterizes an effect on the user from the one or more impacts.

### CROSS-REFERENCE TO RELATED APPLICATIONS

This application claims priority to and the benefit of U.S. Provisional Application No. 63/157,418, filed on Mar. 5, 2021, which is hereby incorporated by reference herein in its entirety.

### COPYRIGHT

A portion of the disclosure of this patent document may contain material which is subject to copyright protection. The copyright owner has no objection to the facsimile reproduction by anyone of the patent disclosure, as it appears in the Patent and Trademark Office patent files or records, but otherwise reserves all copyright rights whatsoever.

### FIELD OF THE INVENTION

The present invention relates generally to systems and methods for calculating an impact score. More particularly, this invention relates to systems and methods for characterizing an amount of potential neurological damage suffered by an athlete during a practice or a game due to one or more impacts experienced by the athlete.

### BACKGROUND OF THE INVENTION

At all levels, athletics are seen as constructive methods of exercise. Sports encourage robust competition and health. Men, women, boys, and girls participate in a variety of sports and athletic activities on a formal and informal basis. Given the variety of individuals involved, there is a large number of activities and sports played by many diverse player types. Some games involve high-speed running. And some involve more physical sports with purposeful or incidental contact between players and/or fixed objects. Contact raises the potential for harm, including head and brain injury. While American football is seen as a primary cause of sports concussions and long-term brain injury, it is less known that players in other sports also experience a high-risk for head injury and brain trauma. For instance, the incidence of concussions in girls' soccer is second only to football, and the combined incidence of concussions for boys' and girls' soccer nearly matches that of football.

Virtually any forceful impact to the head or body involves some risk level for brain trauma. Head injury may occur from collision with another player, an object, or even from a fall. Impact and rotational forces to the head are the leading causes for injury. Brain injury manifests as either neural, or most often, vascular injury within the head.

It is also widely known that the risk and severity of brain injury is related to the frequency and severity of repeated head trauma. A first blow to the head may modify the risk factors for future injury. For instance, a first incidental hit may lower the threshold for injury due to a later fall to the ground. Repeated blows and impacts have a greater impact on the risk of head trauma. Even a minor blow, below the normal threshold for injury, may cause catastrophic brain injury if it follows an earlier risk-elevating first impact. Furthermore, biometric information (i.e., sex, age, height, weight, etc.) provides an additional factor that is needed to determine the impact threshold for predicting brain injury for a particular individual.

During sports play, head injury may manifest as a temporary impairment or loss of brain function. However, more severe concussions may cause a variety of physical, cognitive, and emotional symptoms. Unfortunately, some injuries cause no immediate or obvious observable symptoms, and even minor symptoms may be overlooked, especially during the excited flow of a game. The unknown consequences of prior impacts further exacerbate the risks, by failing to diagnose an injury and take corrective action.

In recent studies, the CDC estimates that about 40% to 50% of athletes will not self-report that they may have suffered a concussive blow. While some portion of these athletes who fail to report head injuries are likely out of stubbornness, the failure to report is often attributed to the player not experiencing traditional or expected concussion symptoms. Consequently, notifying the user that he or she has received a significant impact (or impacts) is necessary for the user to report the event.

The present disclosure provides systems and methods for determining an impact score for the user that characterizes impacts experienced by the user, and provides the user with an estimate of any potential neurological damage due to the impacts.

All these and other objects of the present invention will be understood through the detailed description of the invention below.

### SUMMARY OF THE INVENTION

The present invention is directed to systems and methods for determining an impact score for the user. In one aspect, a method for characterizing impacts experienced by a user comprises receiving a plurality of types of data, determining one or more impact scores for the user based at least in part on the data, and communicating the one or more impact scores to the user. The received data includes impact data and demographic data. The impact data is associated with one or more impacts experienced by the user. The demographic data is associated with the user. The one or more impact scores is based at least in part on the impact data and the demographic data. The one or more impact scores characterize an effect on the user from the one or more impacts.

In one aspect, the impact data includes (i) data related to a linear acceleration of the user associated with the one or more impacts, (ii) data related to a rotational velocity of the user associated with the one or more impacts, (iii) data related to a rotational acceleration of the user associated with the one or more impacts, (iv) data related to an amount of force imparted to the user by the one or more impacts, (v) data related to a duration of the one or more impacts experienced by the user, (vi) data related to a time between successive impacts of the one or more impacts, (vii) data related to a direction of the one or more impacts impact relative to a baseline direction, or (viii) any combination thereof.

In one aspect, the demographic data includes the user's age, the user's sex, the user's height, the user's weight, the user's medical history, a sport that the user plays, a position in the sport that the user plays, or any combination thereof.

In one aspect, the plurality of types of data further includes environmental data associated with an area where the user is located, the environmental data including an ambient temperature of the area, an elevation of the area, a precipitation level of the area, a humidity of the area, an air quality level of the area, or any combination thereof

In one aspect, the one or more impact scores includes a daily impact score, a weekly impact score, a monthly impact score, a rolling average impact score, or any combination thereof. In one aspect, the daily impact score is based on impacts received during a current day and during at least one day prior to the current day. In one aspect, the rolling average impact score is a daily rolling average impact score. In one aspect, the daily rolling average impact score is an average daily impact score across each of seven days prior to a current day.

In one aspect, the method further comprises communicating to the user one or more recommended actions to improve at least one of the one or more impact scores. In one aspect, the one or more recommended actions includes increasing hydration, increasing an amount of sleep, participating in moderate outdoor activity, reducing an amount of time spent by the user viewing an electronic display, a diet change, abstaining from a sport played by the user for a period of time, or any combination thereof.

In one aspect, the one or more impact scores are indicative of a risk of concussion from the one or more impacts, a risk of neurological damage from the one or more impacts, an amount of neurological damage caused from the one or more impacts, a risk of non-neurological damage from the one or more impacts, an amount of non-neurological damage caused by the one or more impacts, or any combination thereof. In one aspect, the impact data is generated using one or more sensors located in a mouth guard worn by the user. In one aspect, the one or more sensors include an accelerometer, a gyroscope, a magnetometer, or any combination thereof.

In one aspect, mouth guard includes one or more light emitting diodes, one or more notification components, or both, and wherein the one or more impact scores are communicated to the user using the one or more light emitting diodes, the one or more notification components, or both. In one aspect, the one or more notification components include a buzzer, a speaker, a piezoelectric element, a magnetic element, or any combination thereof.

In one aspect, a system for characterizing impacts experienced by a user comprises one or more memory devices, and one or more processors. The one or more memory devices have stored thereon machine-readable instructions. The one or more processors are configured to execute the machine-readable instructions to receive a plurality of types of data. The plurality of types of data include impact data associated with one or more impacts experienced by the user, and demographic data associated with the user. The one or more processors are further configured to execute the machine-readable instructions to determine, based at least in part on the impact data and the demographic data, one or more impact scores for the user, the one or more impact scores characterizing an effect on the user from the one or more impacts. The one or more processors are further configured to execute the machine-readable instructions to communicate the one or more impact scores to the user.

In one aspect the one or more processors are located in a mouth guard worn by the user, in a smart device, or both. In one aspect, the impact data is generated using one or more sensors located in the mouth guard. In one aspect, the one or more impact scores are determined using one or more processors of the smart device. In one aspect, the one or more impact scores are determined using one or more processors of the mouth guard.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

The present invention will be described with greater specificity and clarity with reference to the following drawings, in which:

FIG. 1 illustrates a perspective view of the back side of a flexible printed circuit board assembly of an embodiment of the present invention;

FIG. 2 illustrates a perspective view of the front side of the flexible printed circuit board assembly of FIG. 1;

FIG. 3 illustrates one embodiment of a cross-sectional view of the printed circuit board used in the flexible printed circuit board assembly of FIG. 1;

FIG. 4A illustrates a left side profile view of the flexible printed circuit board assembly along the 4A-4A line in FIG. 1;

FIG. 4B illustrates a right side profile view of the flexible printed circuit board assembly along the 4B-4B line in FIG. 1;

FIG. 5 illustrates a schematic of the components of the flexible printed circuit board assembly of FIG. 1 according to one embodiment;

FIG. 6A illustrates a user's stone model that replicates his or her maxillary region and associated dentition, which is used in the process of manufacturing the inventive mouth guard;

FIG. 6B illustrates overlaying a first layer over the user's stone model in the process of manufacturing the mouth guard;

FIG. 6C illustrates embedding the flexible printed circuit board assembly of FIGS. 1-2 in the first layer that overlays the user's stone model in the process of manufacturing the mouth guard;

FIG. 6D illustrates overlaying a second layer over the first layer and the embedded flexible printed circuit board assembly in the process of manufacturing the mouth guard;

FIG. 7 illustrates the finished mouth guard from the manufacturing process of FIGS. 6A to 6D;

FIG. 8 illustrates a graphical user interface on a mobile device that provides an indication of impacts for multiple users of the mouth guard;

FIG. 9 illustrates a flowchart of a method for calculating an impact score for a user;

FIG. 10A illustrates a front view of a first screen of an application executed by a smart device;

FIG. 10B illustrates a front view of a second screen of an application executed by a smart device; and

FIG. 10C illustrates a front view of a third screen of an application executed by a smart device.

While the invention is susceptible to various modifications and alternative forms, specific embodiments will be shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

The drawings will herein be described in detail with the understanding that the present disclosure is to be considered as an exemplification of the principles of the invention and is not intended to limit the broad aspect of the invention to the embodiments illustrated. For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.”

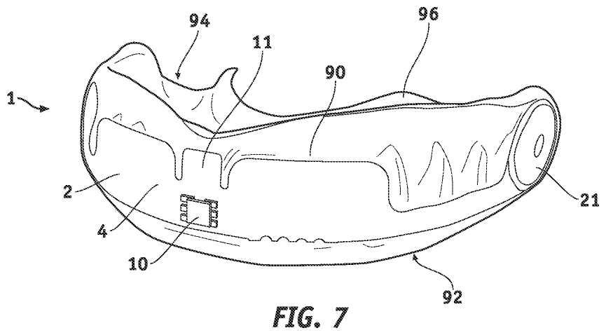

A mouth guard 1, shown best in FIG. 7, includes a flexible printed circuit board (PCB) and electronic components mounted thereon, which define a printed circuit board assembly (PCBA) 2. The PCBA 2 is designed and shaped for placement within the mouth guard 1, which is custom-fit for an athlete's mouth. The electronic components are encased within the flexible material of the mouth guard 1. The PCBA 2 is also flexible and designed with minimal structural dimensions (length, height, width, depth, etc.) for flexing and pivoting to conform to the contours of the mouth guard 1 during manufacturing and use. In one aspect, the invention herein focuses on the novel arrangement of the electronic components on the PCBA 2 to achieve efficient manufacturing, excellent operational performance, and user comfort.

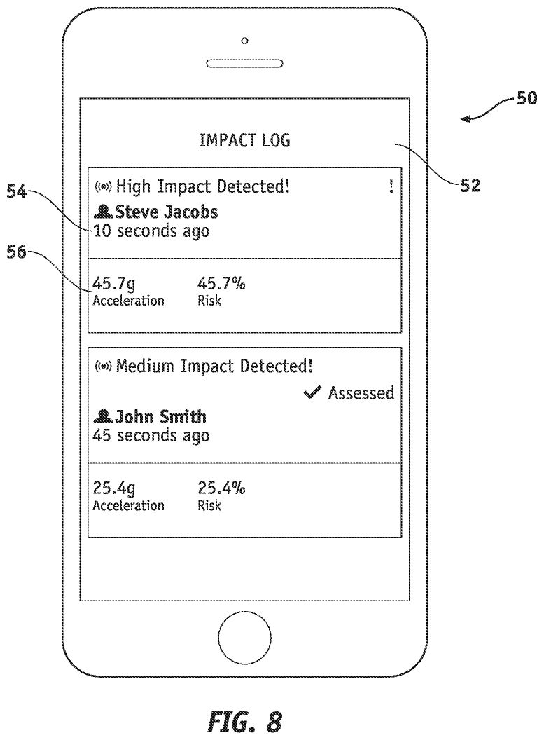

The mouth guard 1 can detect and measure impacts to an athlete's head during sports activities. An array of motion and accelerometer sensors (discussed below) detect and measure an acceleration on the user, which can then be calculated into force data. The impact data is stored on the mouth guard and may be later or contemporaneously transmitted via a transmitter (e.g., a Bluetooth low energy transceiver) to a remote smart device, such as a phone or tablet, or like device (see FIG. 8). Additional information regarding the mouth guard is disclosed in co-owned U.S. Publication No. 2017/0238850 (“Impact Sensing Wearable Device and Method”), which is hereby incorporated by reference in its entirety.

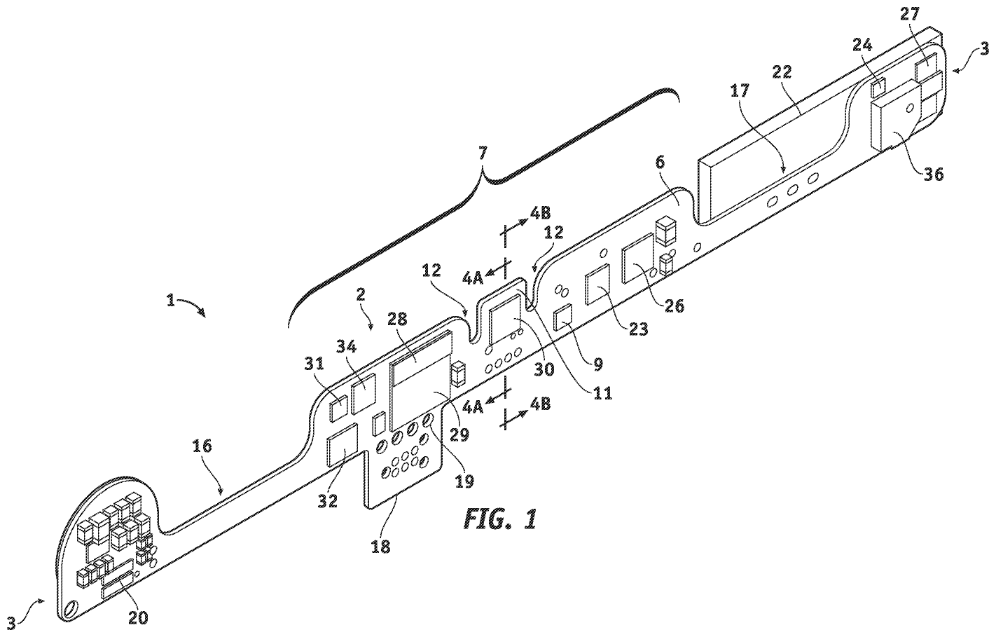

As can be seen in FIG. 1, the PCBA 2 has a length that is defined between two end portions 3 that are intended to rest along the buccal side of the molars. The PCBA 2 includes a front side 4, which will face the buccal region of the wearer after incorporation in the mouth guard 1, and a back side 6 that will face the teeth. A middle portion 7 of the PCBA 2 is positioned between the two end portions 3 and generally has a length between about 25% and 50% of the overall length of the PCBA 2. The electrical components are primarily mounted within the middle portion 7. In one preferred embodiment, the middle portion 7 is about 40% of the overall length of the PCBA 2.

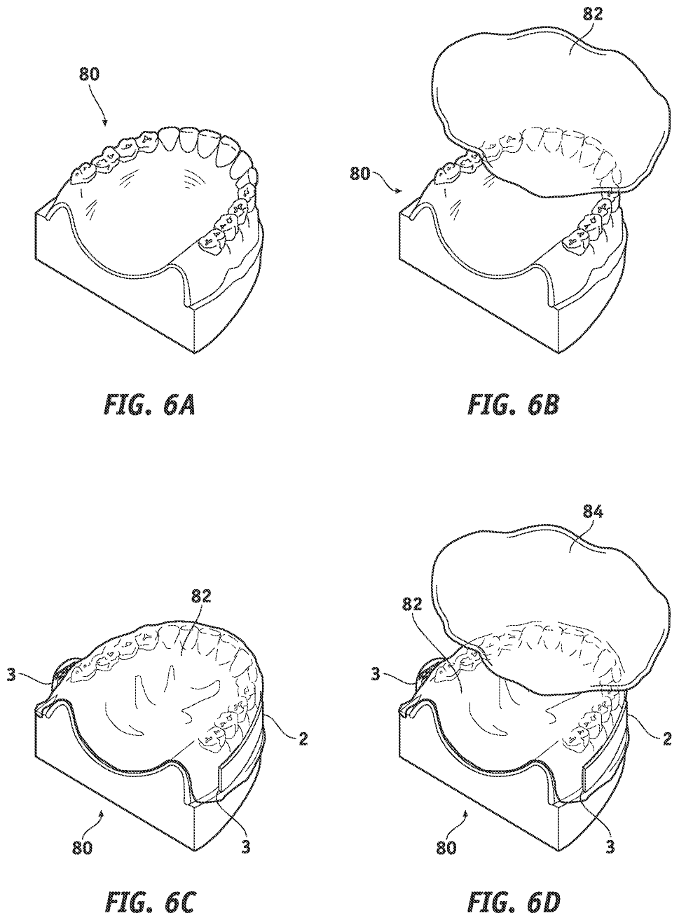

A center tab 11 is located within the middle portion 7 and is positioned in the front center of the mouth near the midline of the user's incisors. The center tab 11 is defined by a pair of cutouts 12 that provide the middle portion 7 with the ability to twist and bend as required during fabrication of the mouth guard 1, as discussed below with respect to FIG. 6. As such, the cutouts 12, which allow for dual axis rotation and twisting at pivot points adjacent thereto, allow the PCBA 2 to overcome the challenges of conforming within the custom mouth guard 1. The two cutouts 12 have a length that is at least 30% of the overall width of the middle portion 7. The middle portion 7 requires the additional bendability because it will be located along the region having the smallest radius of curvature in the dental arch (and, thus, in the mouth guard 1). The bridge portions 16, 17 of the PCBA 2 connect the end portions 3 and the middle portion 7 and are created by larger cut-outs to create a smaller dimension within the bridge portions 16, 17 for enhanced bendability of the PCBA 2.

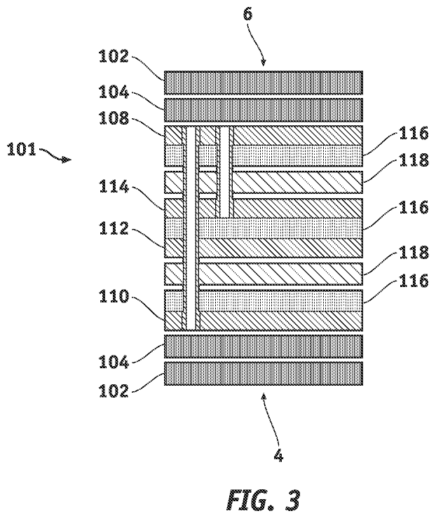

The components are preferably soldered onto PCBA 2 and an underfill (preferably a non-viscous epoxy) is used to fill in spaces within the PCBA 2 to provide some level of rigidity to the PCBA 2. The electrical components on the PCBA 2 are preferably attached with minimal solder. The largest dimension of each of the electronic components is vertically oriented when possible to accommodate bending of the PCBA 2, meaning the long edge is arranged from top-to-bottom while the short edge runs laterally along the length of the PCBA 2. Some components cannot be vertically oriented, such as a wireless charging receiver 20 and a battery 22, and, thus, are preferably placed near the end portions 3 of the PCBA 2, which will be placed along an anatomic region having a larger radius of curvature (i.e., straighter) than the middle portion 7. The PCBA 2 is preferably made of a multilayered board design to act as primary carrier of all electronic components in the mouth guard 1, as discussed below with reference to FIG. 3.

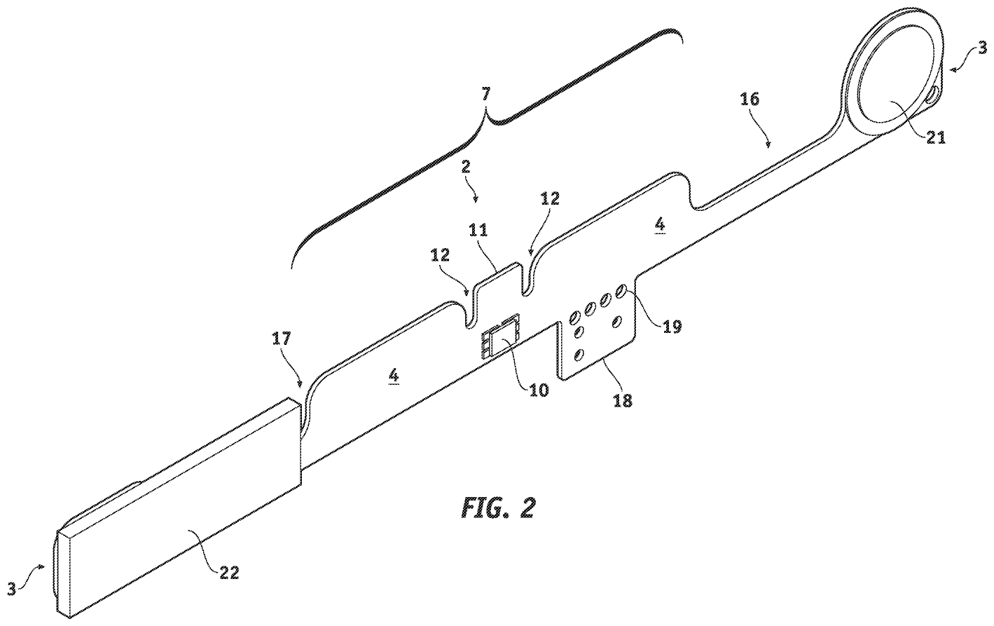

The wireless charging receiver 20 is set on the front side 4 of the PCBA 2. As shown in FIG. 2, the wireless charging coil 21 is set opposite to a charging receiver 20. The charging receiver 20 includes power circuit with inductors and capacitors, and further includes an integrated circuit controller located in proximity to and in electrical communication (e.g., wired) with the wireless charging receiver 20. The wireless charging receiver 20 and the wireless charging coil 2 are preferably set along on of the molar ends 3 and configured to best receive the energy from the external charging station (not shown). As such, the charging coil 21 may be manipulated during the manufacturing process (FIG. 6) to ensure proper positioning so as to minimize skewing relative to the peripheral side surface of the mouth guard 1 that faces the buccal surface.

Regarding the force-sensing components, the PCBA 2 includes a high-G (high-gravity) accelerometer 30, a magnetometer 31 (which includes a digital compass), and a combination low-G (low gravity) accelerometer/gyroscope 32. The magnetometer 31 and the combination low-G (low gravity) accelerometer/gyroscope 32 form an inertial measurement unit (IMU) in that they provide data that is used for sensing the orientation of the mouth guard 1. The data provided by the IMU is utilized in a sensor-fusion algorithm, which is computed in the processor to implement a sensing feature that detects the orientation of the PCBA 2 and the mouth guard 1 in three-dimensional space. The combination low-G (low gravity) accelerometer/gyroscope 32 (hereinafter “gyroscope 32”) can also be provided as two separate components, such that they are not packaged together. Alternatively, the magnetometer 31 can be packaged with the low-G (low gravity) accelerometer/gyroscope 32. The gyroscope 32 also provides data regarding angular velocity (also referred to as rotational velocity), which can be used to determine the angular acceleration (also referred to as rotational acceleration) and angular force (also referred to as rotational force) associated with the impact. Though these sensors 30, 31, 32 are measuring velocity and acceleration, they are herein considered to be linear and rotational force sensors because their sensed data correlates to the corresponding force and permits the processor to calculate it, as necessary.

The approximate location of the impact is computed using the IMU orientation result at the time of the impact and a calculated 3D linear acceleration impact vector. The 3D linear acceleration impact vector (relative to the mouth guard 1) is calculated using the data collected by the high-G accelerometer 30 at the moment of impact. In summary, the data received by the high-G accelerometer 30, the magnetometer 31, and the low-G accelerometer/gyroscope 32 can be used to provide information regarding the amount of linear force associated the impact, the amount of rotational force associated the impact, the spatial orientation of the impact, and movement data of the user.

Data from the high-G accelerometer 30, the magnetometer 31, and the gyroscope 32 is received and processed in a processor 28, which utilizes a memory 34 (preferably a flash memory) to correlate, relate, and otherwise store information for processing and communicating impact data. The memory 34, which may store impact data permanently or be erasable, may be located anywhere on the PCBA 2, but is preferably near the processor 28 to minimize latency. The processor 28 is further coupled with low energy Bluetooth transceiver 29. The Bluetooth transceiver 29 may include a radio and an embedded processor. The processor 28 collects data, stores the data in memory 34, and transmits the data via Bluetooth transmission, as necessary. It should be understood that while a single memory 34 is illustrated on the PCBA 2, it may have multiple memory devices 34. For example, the processor 28 may include its own memory device.

A serial wire debug (SWD) port 18 on the PCBA 2 allows wired access for initial programming of the processor(s) 28. In one embodiment, the initial programming data, such as impact thresholds based on the biometric data of the user, are stored in a memory device associated with the processor 28, while the impact data received by the sensing components is stored in the memory 34 showing in FIGS. 1-2. The user typically provides his or her biometric data when ordering the custom mouth guard 1. The biometric data of the user may include, but is not limited to: age, sex, weight, height, skull circumference, head shape (e.g., from photos of the sides, back, and/or front of the head), head mass (e.g., from a scan of the head), and/or prior concussion data. The biometric data may further include dimensional data taken from the maxilla of the user, such as from a stone model as discussed below with respect to FIG. 6. For example, the combination of the skull circumference along the forehead and the dimensional data of the maxilla provides data that correlates to the mass of the head. Alternatively, a standard mass of the head may be estimated based on the age, sex, weight, and/or height of the user using commonly known biometric information. Assuming that standard mass is known, it can then be adjusted based on measurements of the specific measurements of the user's skull and/or maxilla data. In other words, a user having a skull circumference that is 10% larger than the standard skull circumference for someone of his same age, weight, and height may have the standard mass increased by 5% due to the larger-than-standard skull circumference. Examples of linear and rotational force risk factors and threshold data for different individuals are disclosed in co-owned U.S. Publication No. 2017/0238850 (“Impact Sensing Wearable Device and Method”), which is hereby incorporated by reference in its entirety.

The biometric data of the user is used to set initial thresholds for impact forces (e.g., rotational forces and/or linear forces). In addition to establishing a single maximum threshold over which the risk of a concussion is high, the impact thresholds may include a series-based threshold that takes into account a series of impact events over a certain period of time. The series-based threshold would indicate the risk of concussion due to a series of smaller impact forces (relative to the single maximum threshold) encountered over a period of time. For example, the series-based threshold can be based on a weighted average of the hits, wherein the threshold (as measured by the weighted average) is reduced based on the number of hits. The rate of change in the reduced threshold force may be linear or exponential. These series-based thresholds are a form of dynamic thresholds, in that they change during a session (e.g., a game) of the user's activity, or over multiple sessions of activities. It should be understood that the user's prior concussion history (both short term, such as the impacts occurring over a 24-hour period, or long term, such as a prior concussion within the last two months) is also biometric data of the user that can be used to establish the thresholds. These different initial thresholds values and dynamic threshold values for the user may be stored in various look-up tables in the memory of the PCBA 2 within the mouth guard 1. And, as discussed below in FIG. 8, they may be modified over the course of operation of the mouth guard 1 by the smart device 50.

The user can also indicate different activities for which he or she intends to use the mouth guard 1. For example, a user may indicate that she intends to use the mouth guard for boxing and soccer. Each of these activities may have different impact threshold data that will be stored on the memory device of the PCBA 2 due to the different types of impact forces and frequency of impact forces that are anticipated. Some of the differences may be due to the sensitivity of the impact forces to be detected by the sensors on the PCBA 2. As one example, boxing may have hits that are longer in duration due to the deformation of the boxing gloves, whereas an undesirable head-to-head impact in a soccer match can be of very short duration. Additionally, boxing regulations may restrict certain hits, such as a hit to the back of the head. Because the mouth guard 1 can sense the directionality of hits, a hit to the back of the head in a boxing match, regardless of force, may cause the LED 10 to activate to inform the boxers (and referee) that a restricted hit occurred. Accordingly, the inventive mouth guard 1 may include impact threshold data (e.g., different look-up tables) for multiple activities of the user. The user would use a smart device (e.g., mobile phone 50 in FIG. 8) to communicate the specific activity chosen for that particular day (or session) to the mouth guard 1 via the Bluetooth connection. The processor 28 then pulls the corresponding impact threshold data stored within the memory device for the user's chosen activity and uses that impact threshold data for comparisons during the activity. It should be noted, as explained above, that two different users participating in the same two activities will still have different impact threshold data for each activity (e.g., boxing look-up table #1, soccer look-up table #1, boxing look-up table #2, soccer look-up table #2,) because of their different biometric information. Accordingly, the user's indication of his or her activity or activities (not just biometric information) may also dictate the types of impact force threshold data that are stored in the memory of the PCBA 2.

The various impact thresholds for the user and potentially other data useful for determining concussion risk can be uploaded to one of the memory devices via the SWD debug port 18. Of course, the SWD debug port 18 can be used to upload other data and software into the memory 34. Once the necessary information is loaded on the PCBA 2 and final programming is complete, the SWD debug port 18 can be removed via perforations 19 before it is encased within the flexible material of the mouth guard 1.

Because the high-G accelerometer 30 detects directional impact data, it is preferably located in a predictable reference point and, therefore, is mounted within the center tab 11 such that it is adjacent to the midline of the user's top incisors. Because its physical structure is molded into the mouth guard 1 as discussed below in FIG. 6, the high-G accelerometer 30 maintains this same position along the centerline near the biting or incisive edge line of a maxillary central incisors. In some embodiments with a user having an abnormal, or untraditional, mouth structure regarding tooth placement, fillers may be used in the mouth guard 1 to fill in gaps between teeth (for instance additional material to compensate for missing teeth, misplaced teeth, etc.). Preferably, the electrical components and, in particular, the sensors (e.g., high-G accelerometer 30, magnetometer 31, and gyroscope 32) maintain direct contact with the structure of the mouth guard 1, which, in turn, is molded to maintain direct contact with the skeletal structure and teeth of the head. As such, the sensors maintain indirect (but rigid) contact with the skeletal structure of the head. The sensors can also detect the movement and forces associated with the mouth guard 1 being dropped from the player's mouth or hand so as to discount such impact forces.

An LED driver 9 controls the actuation of an LED 10 on the front side 4 of the PCBA 2. The LED 10 is used to indicate to others (e.g., other players, a coach, a referee) that the user has experienced a certain concussion-risk event (or events when considering a series of impact forces over a period of time). The LED 10 can also indicate operation (i.e., on/off), battery-charge life, malfunction, etc. The LED driver 9 is located on front side 4 of PCBA 2 so that it can be viewed between the user's lips. In a preferred embodiment, the LED driver 9 controls both the light intensity and the color of the LED 10 via the current driven into LED 10. By supplying a fixed current, the LED driver 9 can modify that current to get the appropriate pattern of light(s) displayed on the LED 10 to indicate certain information to others located around the user (and to the user when he or she removes the mouth guard 1 from his or her mouth).

The battery 22 typically supplies between 3 volts and 4.2 volts. To maintain adequate performance levels of components, a main voltage converter 26 is used to provide a constant voltage to the components. A battery charger 23, which is coupled to the wireless charging receiver 20, preferably includes an integrated circuit to monitor and provide a specific charging profile for the battery 22. The wireless coil 21 receives alternating current, and converts it to direct current for the battery 22. The wireless coil 21 preferably receives alternating current at approximately one million hertz, or as otherwise known in the art.

The PCBA 2 includes a battery fuel gauge 24 near one of the end portions 3. The battery fuel gauge 24 utilizes a Coulomb-countering feature and a comparative table to calibrate the charge remaining on the battery 22. The battery 22 typically operates in multiple modes, such as in a normal operational mode, a charging mode, and a standby mode. The battery 22 is preferably a Lithium polymer battery with a low-profile and an ability to slightly bend during the manufacturing process (discussed in FIG. 6) to foster an ergonomic fit into the mouth guard 1. In one preferred embodiment, a gel electrolyte lithium battery is preferred, which most preferably uses a non-toxic electrolyte. While lithium polymer batteries are preferred, as battery technology improves and/or changes, other types of batteries having a thin profile and a light weight (and preferably the non-toxic nature of the electrolyte) will drive selection of battery. A battery protection component 27 may be used to prevent shorting of the battery via overcharging and/or undercharging. The battery protection 27 also prevents the battery 22 from running below a minimum charge. Because of the size of the battery 22 (length and height dimensions), the battery 22 is located toward one of the end portions 3 of the PCBA 2 in which there is more space between the buccal surface and the bone/teeth. Advantageously, the mouth guard 1 has a larger radius of curvature in this region as well, which results in less bending of the battery 22.

The PCBA 2 includes a wearer notification component 36, preferably adjacent to the end 3 of the PCBA 2. The notification component 36 uses electrical energy to generate mechanical energy to provide feedback that can be physically sensed, such as haptic feedback, vibratory feedback (e.g., a buzzer), auditory feedback (e.g., a speaker), or any combination thereof. The feedback can be heard by the ear and/or felt within the mouth. The notification component 26 may include magnetics and/or piezoelectric elements. Because of the generation of mechanical and/or audible energy, the notification component 36 may be one of the high-energy consumption components on the PCBA 2 (the LED 10 is often the highest energy consuming component, depending on the size and functionality). Furthermore, the notification component 36 typically has the tallest profile rising from the PCBA 2 and uses a fair amount of three-dimensional space extending off of the surface of the PCBA 2. To create the most space for the notification component 36, the notification component 36 is located toward the end portion 3 of the PCBA 2 in which there is more space between the buccal surface and the bone/teeth. The notification component 36 is preferably positioned on the back side 6 of the PCBA 2 directly adjacent to the bone just below the maxillary sinus cavity to vibrate the bone and conduct vibrations along the maxilla toward the ear, which may provide a tonal sensation and/or an audible sensation (depending on the vibration frequency).

In other embodiments, the notification component 36 may use an air vibration conductor via a magnet and plate using compressed air. Alternatively, the notification component 36 may create haptic feedback through motors using offsetting weights. Alternatively, the notification component 36 may include a linear resonant actuator (LRA) using a small metal block, or pin.

The notification component 36 is particularly useful to notify the user that he or she has received a significant impact (or series of impacts) that he or she may not have recognized. For example, after a single, high-force impact creates a substantial risk of concussion, the processor 28 receives the data from the rotational and/or linear force sensing units and determines whether the predetermined threshold has been exceeded. If so, the processor 28 then communicates with the notification component 36 to begin activation that results in the haptic feedback, a vibratory feedback (e.g., a buzzer), or auditory feedback. In some embodiments, the processor 28 may delay the activation of the feedback by a set period of time (e.g., 10 or 20 seconds) such that the user has some time to regain full awareness after a big hit, so as to understand what the feedback is intended to mean. The notification component 36 can also provide different types of feedback (in duration, magnitude, or frequency) to inform the user of different events. For example, a series of lesser hits within a period of time that causes the series-based threshold to be exceeded may have a different feedback (e.g., smaller magnitude and a lower frequency) than a single, high-force impact of the component (e.g., high magnitude and a high frequency, or constant feedback). The notification component 36 can also communicate other events, such as a low-battery mode or to remind the user that system is operational, which may be accomplished in a single subtle feedback at a very low frequency (e.g., every 60 seconds).

As seen in FIGS. 1 and 2, the vast majority of the components electronics are within the middle portion 7 of the PCBA 2, but most are to the side of center tab 11 except for the LED 10 and the high-G accelerometer 30. Further, the vast majority of the sensitive electrical components are on the back side 6 of the PCBA to allow them to be impressed into the flexible material of the mouth guard 1 in a direction that faces the teeth to give them added protection from impacts to the face. As noted above, the LED 10 is on the front side 4 so as to permit the viewing of the LED 10 by other people around the user. The battery 22 is likewise on the front side 4 of the PCBA 2 for spatial reasons. The charging coil 21 is on the front side 4 to provide better access for charging the battery 22. FIGS. 1-2 represents one embodiment of the present invention and the components can be rearranged on the PCBA 2.

The width of the of the PCBA 2 (top-to-bottom) varies along the length and is between 2 mm and 15 mm, with the largest width being in the end region 3 in the illustrated embodiment. The middle portion 7 has a height in the range of 6 mm to 15 mm, and is preferably about 12 mm. Each of the end portions 3 having a width in the range of 8 mm to 15 mm. The bridge portions 16, 17 have a width that is smaller than 40% (and preferably smaller than 30%) of the widths of the end portions 3. The bridge portions 16, 17 have a width that is smaller than 50% (and preferably smaller than 40%) of the width of the middle portion 7.

FIG. 3 is a general schematic in cross-section of one preferred embodiment for a flexible printed circuit board (PCB) 101 that would receive the electrical components and form the PCBA 2. All layers are shown in FIG. 3, though not all of them are present across the entire PCB 101 as understood by those skilled in the art. Unlike prior art rigid PCBs that include foil stacked on glass or other rigid materials, the flexible PCB 101 is preferred as its shape may be modified and bent into an ergonomic position, as discussed more in FIG. 6. The flexible PCB 101 has multiple conductive layers, such as four metallic traces, that transit the signals between the electrical components. An upper and lower overlay surfaces 102 protect those internal layers, but would not present in areas when component soldering is required. Two solder-resist layers 104 are located on the metallic (e.g. copper) trace 108 of the back side 6 (primary component side) and the metallic trace 110 on the front side 4, at least in regions of the PCB 101 where component soldering is needed. The electrical working layers further include a ground layer 112 and a power layer 114 (or signal and power layer 114). Each of the four metal layers 108, 110, 112, and 114 is about 0.02 mm in thickness (e.g., 0.018 mm or 0.022 mm).

The ground layer 112 and the power layer 114 is separated by a dielectric layer 116, such as polyimide. The back side copper trace 108 is separated from the power layer 114 by a dielectric layer 116 and an adhesive layer 118. The front side copper trace 110 is also separated from the ground layer 112 by a dielectric layer 116 and an adhesive layer 118. The dielectric layers 116 and adhesive layers 118 are each about 0.025 mm in thickness. The overall thickness of the PCB 101 is between about 0.2 mm and about 0.3 mm. The PCB 101 may include exterior side tape on both the front side 4 and back side 6 in some regions, which is useful in mechanically attaching some of the larger components (e.g., the battery 22 and the charging coil 21) to the PCB 101.

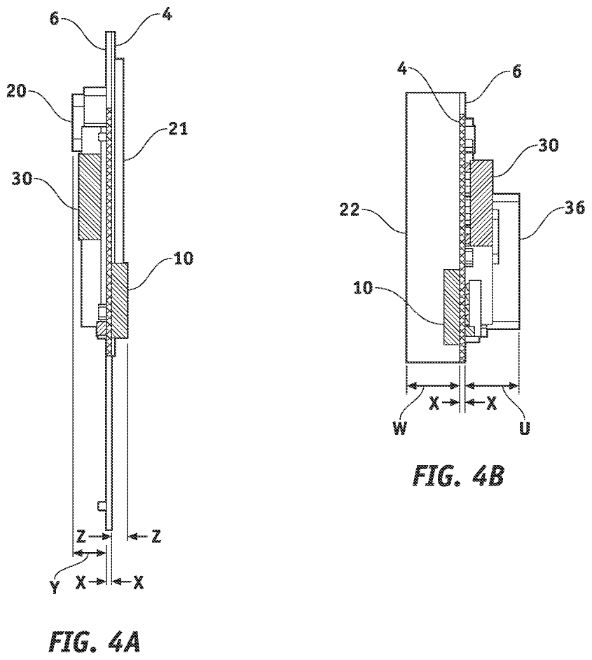

FIGS. 4A and 4B illustrate the left side and right side cross-sections of the PCBA 2 along, respectively, lines 4A-4A and lines 4B-4B of FIG. 1, both of which cut through the high-G accelerometer 30 on the back side 6 and the LED 10 on the front side 4. The dimension X-X refers to the overall thickness of the printed circuit board, which is about 0.2 mm in the illustrated embodiment. The dimension Z-Z in FIG. 4A references the height of the LED 10, which is about 0.6 mm. The battery 22 shown in FIG. 4B has a height dimension W of about 2.0 mm. The notification component 36 shown in FIG. 4B also has a height dimension U of about 2.0 mm. The charging coil 21 shown in FIG. 4A has a height dimension of about 0.4 mm, which is less than the height dimension of the LED 10. One of the parts of the coil charging receiver 20 has a height dimension Y-Y in FIG. 4 of about 1.2 mm. According, while the overall PCBA 2 has a length of about 110 mm, the maximum overall height dimension of the PCBA 2 (including components on the top side 4 and read side 6) is less than 5 mm in all regions, such that the ratio of length to overall height is greater than 20.

The end portion 3 with the battery 22 and notification component 36 may have a maximum overall height dimension of about 4.2 mm, and is preferably less than 4 mm. In the middle portion 7, the maximum overall height dimension at any point along the length is less than 2 mm (e.g., 1.8 mm), and is preferably less than 1.5 mm, which is dictated by the nearly overlaying the high-G accelerometer 30 on the back side 6 and the LED 10 on the front side 4. In the end portion 2 with the coil 21, the maximum overall height dimension is less than 2 mm (e.g., 1.9 mm), and is preferably less than 1.5 mm. The bridge portions 16, 17 of the PCBA 2 that connect the end portions 3 and the middle portion 7 preferably include no components and have a maximum dimension between about 0.2 mm to 0.3 mm, and preferably about 0.2 mm (i.e., the thickness of the printed circuit board in FIG. 3) to provide additional flexibility (both bendability and rotational twistability) to the overall PCBA 2, which is helpful when manufacturing mouth guard 1 as shown in FIG. 6. FIGS. 4A and 4B illustrate only one exemplary preferred embodiment of a low-profile PCBA 2 for use in the mouth guard 1, as other component arrangements and configurations can be used.

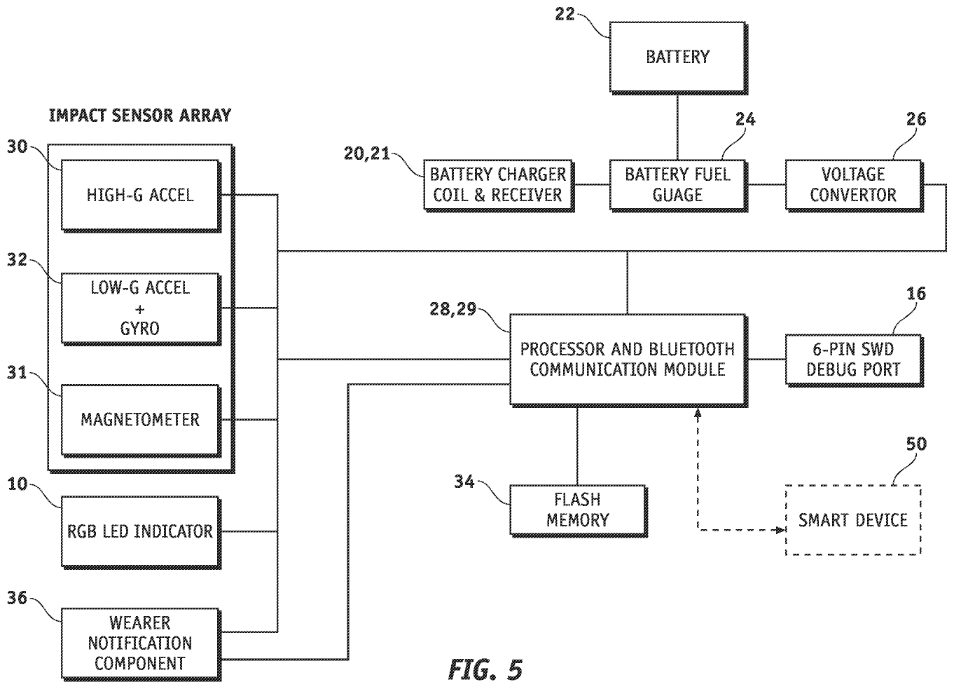

FIG. 5 is one embodiment of a general schematic illustrating the connectivity of the components mounted on the PCBA 2. The battery 22 provides power to all of the various components. The processor module includes the main processor 28 in communication with the components, receiving data (e.g., from the sensors) and sending communications instructions (e.g., to the LED 10 and notification component 36). The processor module may also contain the Bluetooth transceiver 29 that allows for communication with one or more external devices, such as the smart device 50 associated with of the user, a parent, a coach, a referee, etc. The smart device 50 would include the software (e.g., in the form of an app) to receive and transmit information with the Bluetooth transceiver 29 within the mouth guard 1 while in use or before/after use. The smart device 50 also provides remote storage, computation, and display of risk factors associated with one or more mouth guard devices. The smart device 50 may be smart phone, a tablet, or a computer. One exemplary smart device 50 is shown in FIG. 8. Other system configurations for the connectivity of the components of the PCBA 2 are possible as well.

FIGS. 6A-6D illustrate a series of stages in developing the mouth guard 1. A model 80 of the user's mouth, specifically of the maxillary region teeth, is made. The model 80 may be created by a dental professional, by use of an intra-oral scanner, or by use of a simple home impression tray. As showing in FIGS. 6A-6D, the model 80 is a stone model that is formed within an impression that was impressed over the user's teeth and gingiva. The stone is poured into the impression to create the stone model 80, resulting in a replica of the user's upper teeth and bone structure.

In one preferred embodiment shown in FIGS. 6A-6D, the method for forming the wearable mouth guard 1 is performed as follows. As shown in FIG. 6A, the model 80 is placed upside down and inserted into a thermoforming machine (e.g., a pressure thermoforming machine) that is designed to produce custom mouth guards, such as a Drufomat® manufactured and sold by Dentsply Sirona. As shown in FIG. 6B, a first base layer 82 of about 3 mm of EVA thermoplastic (other flexible materials are possible as well) is placed over the model 80. Within the thermoforming machine, the first layer 82 is heated and thermoformed over the model 80 so that it will substantially conform to the anatomical structures (e.g., bone, gingiva, teeth, etc.) of the maxillary region of the oral cavity of user, as dictated by the model 80. Due to the heat and pressure, the initial thickness of the first layer 82 decreases by about 30% to 40% when overlaid onto the model 80. For example, when the first layer 82 of 3 mm is used, the final thickness of the first layer 82 is approximately 1.8 mm to 2.1 mm. The first layer 82 is then ready to receive the PCBA 2. It should be noted that trimming and polishing may be performed on the first layer 82, as necessary.

The PCBA 2 is tested and programmed prior to being molded into the base layer 82. Once the PCBA 2 passes all tests and the latest firmware has been programmed, the region with the SWD debug port 18 (FIGS. 1-2) is trimmed off using scissors along the perforations 19. By use of the debug port 18, the memory 34 is programmed to include, for example, the specific predetermined threshold data points based on the biometric information received from that particular user.

Once the programmed PCBA 2 is ready, a heat gun is employed to sweep across and soften the first layer 82. In one embodiment, a 700 W heat gun with about 120 L/m of air flow is set to 350° C. and is used for about 30 seconds to heat the material. With the first layer 82 now softened, the PCBA 2 is placed with the back side 6 (and the majority of the electrical components) facing inward towards the softened first layer 82. The softening is not harsh enough to affect the conformance of the first layer 82 to the underlying model 80. For the first connection point, the high-G accelerometer 30 on the central tab 11 (not shown in FIG. 6) is positioned directly adjacent to the midline of the two central incisors. The central tab 11 and physical structures of the high-G accelerometer 30 and adjacent components, such as the LED driver 9 and the processor 28, on the rear side 6, are forced into and depressed within the first layer 82. For vertical alignment, the edge of the middle portion 7 closest to the LED 10 should be aligned with the cutting edges of the replica incisors within the model 80. Next, the remainder of the components on the rear side 6 of the middle portion 7 are impressed into the softened first layer 82.

After the components of the middle portion 7 are impressed within the first layer 82, the remaining portions of the left side and right side of the PCBA 2 are depressed into the first layer 82, including all components (e.g., wireless charging components 20 and the notification component 36) near the two end portions 3. Typically, the heat gun may be needed again to soften the first layer 82 along its sides to accommodate those materials. The bridge portions 16, 17 of the PCBA 2 provide the bending and twisting needed to accommodate the wide variety of anatomic variations in the maxilla region of the general population. As shown in FIG. 6C, the PCBA 2 is fixed into the first layer 82 by impressing the geometric arrangement of the electrical components on the back side 6 of the PCBA 2 into the softened first layer 82. The LED 10, the battery 21, and the charging coil 21 are mounted to the front side 4 of the PCBA 2 and, thus, project outwardly away from the underlying model 80.

Because of the unique shape of the model 80 due to each user's unique anatomy, the PCBA 2 will fit a bit differently on the first layer 82 for each user. Accordingly, a deformable filler material, such as Fillin™ from Dreve Dentamid GmbH, is provided to fill in the small gaps between the back side 6 of the PCBA 2 and the first layer 82, and also between the first layer 82 and some of the larger inwardly-facing (tooth-facing) components, such as the wireless charging components 20 and the notification component 36. This deformable material is similar to a wax such that it can be rolled into thin pieces and shaped as necessary. Additionally, the deformable filler material is used to smooth any rough surfaces (like the corners of the battery 22 or the coil 21) to prevent unnecessary pressure points for the next layer. This helps to eliminate gaps between the PCBA 2 (and the fronts-side electrical components) and the base layer 82, which may permit air to become trapped when the second layer 84 (e.g. EVA) is applied. In other words, the deformable filler material is used to fill in gaps and smoothen sharp corners for a more reliable placement of the second layer 84. The deformable filler material can also be type of hot glue, such as EVA.

As shown in FIG. 6D, the second layer 84 is placed over the combination of the first layer 82 and the PCBA 2 (and any Fillin™ material) within the thermoforming machine, to allow pressure molding of the second layer 84 over the PCBA 2. The heated second layer 84 is formed to fit over the LED 10, the battery 22, and the charging coil 21 on the front side 4 of the PCBA 2. The second layer 84 can be anywhere from 1 mm to 4 mm and, like the first layer 82, becomes thinner (and denser) upon application of heat and pressure. Assuming a 2 mm final thickness of the first layer 82, a second layer 84 of 1 mm creates about 3-4 mm thickness across most of the mouth guard 1, which may be more appropriate for sports like biking, basketball, etc. Assuming a 2 mm final thickness of the first layer 82, a second layer 84 of 4 mm creates about a 4-5 mm thickness across most of the mouth guard 1, which may be more appropriate for direct impact sports, such as boxing or football. The thickness of the second layer 84 depends in part on user preference, and the requirement for safe functionality. Trimming and polishing can be performed after the second layer 84 has been added to the first layer 82 and PCBA 2.

The total thickness, of course, varies a bit over the regions of the mouth guard 1 due to the electrical components on the PCBA 2. For example, in the front portion 92 (FIG. 7) of the mouth guard 1 having an arch-shaped surface 94 (FIG. 7) exposed to the buccal surface, the end portion 3 of the PCBA 2 having the battery 22 and notification component 36 will have the greatest localized thickness of the overall mouth guard 1, with a thickness less than 7 mm, and preferably less than 6 mm. In the front portion 92 of the mouth guard 1 exposed to the buccal surface and near the middle portion 7 of the PCBA 2 (with the majority of the components), the maximum localized thickness of the mouth guard 1 will be less than 5 mm, preferably less than 4 mm. In the front portion 92 of the mouth guard 1 exposed to the buccal surface near the other end portion 3 of the PCBA 2 having the charging coil 21 and wireless charging components 20, the maximum localized thickness of the mouth guard 1 will be less than 5 mm, preferably less than 4 mm. Between these three regions, the thickness in the bridge portions 16, 17 be less than 4 mm, or less than 3 mm.

In one example illustrating the thickness of the front portion 92 of the mouth guard 1 that faces the buccal region of the user's mouth, with the first layer 82 being 3 mm in thickness and the second layer 84 being 2 mm in thickness, the end portion 3 with the battery 22 has a maximum thickness of about 5.5 mm, the middle portion 7 has a maximum thickness of about 3.0 mm, and the end portion 3 with the charging coil 21 has a maximum thickness of about 3.3 mm. In a second example illustrating the thickness of the front portion 92 of the mouth guard 1, with the first layer 82 being 3 mm in thickness and the second layer 84 being 3 mm in thickness, the end portion 3 with the battery 22 has a maximum thickness of about 6.1 mm, the middle portion 7 has a maximum thickness of about 3.7 mm, and the end portion 3 with the charging coil 21 has a maximum thickness of about 4.0 mm

The overall thickness of the mouth guard 1 should not necessarily impact the data measured, but can be compensated for by algorithms or software when programming the processor 28 for the predetermined threshold levels. For example, when additional thickness of the mouth guard 1 is used, it dampens the measured impact force. In other words, the mouth guard 1 is effectively reducing the amount of impact/force on the skull and brain by a small amount.

FIG. 7 demonstrates the finished mouth guard 1 produced by the manufacturing process of FIG. 6. The mouth guard 1 includes a main body having the arched-shaped peripheral side surface 90 on its front portion 92 that generally follows the contour of the user's dental arch in the maxilla region. The front portion 92, which is comprised of the layering of the first layer 82, the PCBA 2, and the second layer 84, includes the electronic components. While most of those electronic components are facing inwardly toward the teeth (and not seen in FIG. 7), but the LED 10 faces the buccal direction and can be seen through the second layer 84 of plastic. The charging coil 21 is also seen on the front side 4 of the PCBA 2. The center tab 11 that receives the high-G accelerometer 30 is positioned in a location that is substantially aligned with the midline of the upper incisors of the user. The mouth guard includes an arch-shaped depressed portion 94 connected to the front portion 92 that is sized and shaped to receive teeth of the user to help retain the mouth guard in a fixed position in the mouth. A palate portion 96 of the mouth guard 1 engages the roof of the mouth. Given the drive to produce a device that minimizes the size, permits accurate data collection, and provides user comfort, the mouth guard 1 of the present invention works better with the aforementioned forming process. Though it is possible to use a mouth guard created from a one-size-fits-all approach (such as boil-and-bite mouth guards) with the present invention, the aforementioned forming process is the preferred method of manufacturing.

The first layer 82 can be made of a clear or colored material. The second layer 84 is preferably clear in the region of the LED 10 so that it can be observed by others, but other regions of the second layer 84 can be colored. If no LED 10 is present in the mouth guard 1, then both layers 82 and 84 can be colored and opaque.

As shown in FIG. 8, a remote receiver in the smart device 50 may act with a local app (e.g., software application) to receive and display real-time data or information from the mouth guard(s) 1 via the communication system (e.g., Bluetooth transceiver 29). Preferably, the remote smart device 50 includes a display 52 that permits the displaying of certain types of impact information from a specific mouth guard 1 associated with a user (or multiple mouth guards from several users). For example, the display 52 may include a relative time indicator 54 that may demonstrate the timing of a recent impact event. Furthermore, the display 52 may include impact data 56, such as acceleration and force (linear and/or rotational). Further, a risk calculation 58 may be displayed. The risk calculation may be determined either locally or remotely, and is based predetermined impact thresholds unique to each user based on his or her biometric information. The risk calculation can also be dynamically generated in that it is based on series of impacts within a certain period of time. Accordingly, the present invention contemplates that when the mouth guard 1 senses a force exceeding a certain threshold, there are multiple options for notifications: (i) to the user via the feedback from the notification component 36, (ii) to other individuals near the user via the LED 10, and (iii) to more-remotely situated individuals via the display 52 of the smart device 50.

As noted above, the smart device 50 also permits the user to communicate with the mouth guard 1. For example, the user can use the smart device 50 to select his or her activity for the day such that processor 28 then selects the corresponding impact force threshold data for that particular activity. The user can use the smart device 50 to indicate the occurrence of a prior concussion, such that processor 28 then selects a reduced impact threshold data. Or, in another alternative, the user can dictate the reduced value of the impact threshold by indicating a specific percent reduction in the threshold if he or she desires to be notified of lesser hits to stay far away from injury. The smart device 50 may, in response to the user's command, download force data from memory 34 on the PCBA 2, and then send instruction to erase the prior force data or segments of the prior force data.

The smart device 50 is preferably in communication with a remote storage (e.g., the cloud) that contains the various types of threshold data for all users or a subset of users (e.g., football players). As better and more precise threshold data is learned and stored in the remote storage, the smart device 50 downloads that updated data, which can then be transmitted to the user's mouth guard 1 via the Bluetooth connection. In another example, if the user decides to try another activity, such as rugby, the smart device 50 can download the rugby threshold data from the remote storage and then transmit that rugby threshold data to the customized mouth guard 1 for that user.

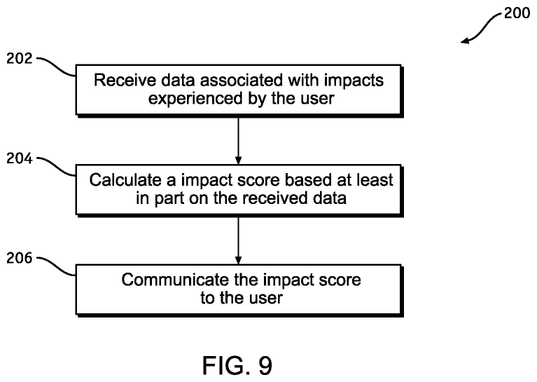

FIG. 9 illustrates a flowchart for a method 200 of calculating one or more impact scores for a user. The user's impact score is a measure of one or more impacts that the user has experienced, and provides a point of reference for the user to determine how much strain, stress, fatigue, etc. that the user is experiencing or has experienced due to the one or more impacts. In some implementations, the method 200 can be performed using one or more processing devices. The one or more processing devices can include only the smart device 50, the smart device 50 in combination with the smart device 50, or only processing device(s) other than the smart device 50.

At step 202 of the method 200, data used for calculating the impact score is received. The received data can include a plurality of different types or categories of data that may be useful for calculating the impact score. A first category of data can be impact data associated with any impacts experienced by the user. In some implementations, some or all of the impact data is generated by the mouth guard 1. For example, the impact data can include any data that may be generated by the high-G accelerometer 30, the magnetometer 31, and/or the low-G accelerometer/gyroscope 32, which can include a variety of different position, velocity, acceleration, and/or force data. The impact data can also include any impact data calculated or derived from the data generated by the high-G accelerometer 30, the magnetometer 31, and/or the low-G accelerometer/gyroscope 32. For example, the impact data can include data that specifically characterizes the severity of the impacts, which may itself be based on the data generated by the high-G accelerometer 30, the magnetometer 31, and/or the low-G accelerometer/gyroscope 32.

The impact data can generally include data related to a linear and/or rotational velocity of the user associated with any impacts, a linear and/or rotational acceleration of the user associated with any impacts, a linear and/or rotational acceleration of the user associated with any impacts, the number of impacts experienced by the user, a force imparted to the user by any impacts, the duration of any impacts experienced by the user, the time between impacts experienced by the user, the direction of the impact relative to a baseline direction (e.g., the direction that the user is facing at the time of the impact), an elapsed time since the last impact, or any combination thereof. Generally, the impact data is kinematic data (e.g., data related to the motion of the user associated with impacts). However, the impact data can also include non-kinematic data that is associated with any impacts.

In some implementations, the impact data can also include data related to the location on the user where the impact occurred. The orientation of the user's head at the time of impact and the movement of the user's head due to the impact can be determined based at least in part on data from the high-G accelerometer 30, the magnetometer 31, and/or the low-G accelerometer/gyroscope 32. The location on the user where the impact occurred can then be determined based at least in part on the orientation of the user's head and the movement of the user's head due to the impact.

In other implementations, the impact data can include any data relevant to impacts experienced by the user that do not ultimately stem from the mouth guard 1. For example, in some implementations image data and/or video data can be generated that is reproducible as an image or a video of an impact experienced by the user. The image data and/or the video data can be analyzed to determine the location on the user where the impact occurred, in addition to or as an alternative to determining the impact location using the high-G accelerometer 30, the magnetometer 31, and/or the low-G accelerometer/gyroscope 32. Thus, in some implementations, the received data includes image data and/or video data associated with an impact experienced by the user.

A second category of data can include demographic data. The demographic data can generally include any personal information about the user that may be used to calculate the impact score. In some implementations, the demographic data includes biometric data associated with various biological characteristics of the user, such as the user's age, the user's sex, the user's height, the user's weight, medical data, etc. The medical data can include data indicating whether the user has previously suffered from any neurological injuries (such as a concussion), or any non-neurological injuries (such as broken bones, torn muscles, etc.). The medical data can also include data indicating whether the user has ever participated in practices, games, events, etc. while suffering from any neurological injuries or non-neurological injuries.

The demographic data can also include non-biometric data, such as the sport the user is playing, the position that the user plays in the sport, historical data related to the user's participation in the sport (which could include data about previous impacts, events, etc.), family history data, etc. In some implementations, the demographic data can be formatted as absolute data. For example, the demographic data could indicate that the user is an 18-year old male, is six feet two inches tall, and weighs 210 pounds. In other implementations, the demographic data can be formatted as relative data. For example, the demographic data could indicate the user the is in the 80^{th} weight percentile for a user of their age and sex.

In some implementations, the demographic data can include user-inputted answers to questions related to how the user's feel. For example, the user can be asked if they have a headache, how their energy levels feel, whether their heart rate is elevated, whether their mind feels sharp, etc.

A third category of data can include environmental data related to the geographical area in which the user is located when the user experienced the impacts in questions. For example, the environmental data can include the ambient temperature, elevation, precipitation, humidity, air quality, etc. The environmental data can also include the user's location, for example in the form of a zip code, an area code, a town, a county, a state, latitude and longitude coordinates, etc. In some implementations, the user's location can be used to calculate, estimate, or infer other environmental data, include the temperature, elevation, precipitation, humidity, air quality, etc.

In some implementations, the received data can represent the duration and quality of the user's recovery from prior impacts. For example, the data can generally include the amount of time elapsed since the last impact (which could, for example, be calculated based on timestamps of the impacts), as well as data related any actions undertaken by the user since the last impact, such as sleep, exercise, recovery activities, hydration levels, diet, etc.

The data can be generated and/or collected from a variety of different sources. In some implementations, a portion of the data can be generated from mouth guard 1 and/or any of the sensors included therein. In some implementations, a portion of the data can also be collected from publicly available sources, such as government databases, non-profit databases, etc. In some implementations, portion of the data can also be collected from third-party sellers of data. In some implementations, data from other users of the mouth guard 1 can be collected and stored, so that any given user can access the other user data for a variety of purposes, including comparing their own impact scores and impact counts.

In some implementations, the received data includes data that is input by the user. For example, using any of the one or more processing devices, the user can input any data that might be relevant to calculating the impact score. In some implementations, the user can input data related to impacts that the user has experienced, such that the impact data includes data received directly from the user. The user can also input any relevant demographic or environmental data.

At step 204 of the method 200, the impact score is calculated based at least in part on the received data. The impact score can provide the user with an indication of the stress or strain that the user has undergone due to the impacts they have experienced, so that the user and/or other people (parents, coaches, trainers, doctors, etc.) can determine the best course forward for the user following the impacts (e.g., sitting out the next practice or game, getting more rest). In some implementations, the impact score characterizes potential neurological damage to the user (e.g., concussions or other brain-related damage). In these implementations, the impact score may include a determined percent chance that the user experienced a concussion during the time period for which the impact score is measured. In other implementations, the impact score characterizes potential non-neurological damage, such as bruises, broken bones, etc. In still other implementations, the impact score characterizes both neurological damage and non-neurological damage.

The impact score can be based on any of the data received at step 202, so as to provide the user a personalized estimate of their own strain from the impacts. In some implementations, the impact score accounts for biological differences in users to characterize the effect of impacts. For example, an impact that imparts a given force on a smaller user may be more detrimental than the exact same impact when experienced by a larger user. In another example, an impact imparts a given force on a user who has little to no history of experiencing such impacts may have less of a negative effect on that user as compared to a user who has a long history of experiencing such impacts.

The impact score can correspond to the strain the user experienced during a variety of different time periods. In some implementations, the impact score is a daily impact score, and characterizes the impacts the user has received during a single day. In other implementations, the impact score is a weekly impact score. In still other implementations, the impact score is calculated for a single event corresponding to the user's sport, such as one practice, one game, one event in a multi-event meet (e.g., a swim meet or a track and field meet). At the end of the relevant time period, the impact score can reset and be newly calculated for the next time (e.g., the next day, the next week, etc.). In still other implementations, the impact score is continually calculated over a rolling time interval, and thus is not re-set at the end of the time interval. In these implementations, the strain is score is generally re-calculated on a second and shorter time interval. For example, the impact score may be calculated for the previous 24 hours, and be updated every hour. The rolling time interval could be a day, a week, a month, or other time intervals. These impact scores can be referred to as rolling average impact scores. In some implementations, multiple impact scores can be calculated for the user. For example, a daily impact score can be calculated each day (e.g., the daily impact scores resets at the end of the day), along with a weekly rolling average impact score that is updated each day.

In still other implementations, the impact score used to characterize impacts experienced during a first time interval can take into account impacts experienced during a second time interval. For example, when calculating a daily (first time interval) impact score, impact data associated with impacts experienced by the user over the prior week (second time interval) can be taken into account in calculating the impact score. Thus, a user who experiences a given amount of impacts during the current day and has experienced few impacts during the prior week can have a lower impact score than a different user who experiences the same amount of impacts during the current day, but has experienced a greater number of impacts during the prior week. The first time interval and the second time interval can be selected as required for the analysis. The first time interval and the second time interval could be one day and one week; one hour and one day; one week and one month; and others. The time intervals could also correspond to specific events, such as games, practices, etc.

In some implementations, the impact score is calculated in absolute terms. In other implementations, the impact score is calculated in relative terms, and thus acts as a comparison between the user and some other metric or target. In some implementations, the impact score is calculated as the user's strain relative to other user's in the same population. The population could include all other users who play the same sport and the same position as the user, all other users having a height and weight within predetermined bands. Generally, any number of different biometric and non-biometric qualities can be used and/or combined to form the relevant population.

In some implementations, the impact score can be calculated relative to previous impact scores for the same user. For example, a baseline amount of strain can first be established. The baseline amount of strain may be the average strain the user experiences in one day, during one practice, during one game, etc. Then moving forward, the impact score can be indicative of the amount of strain that the user experienced relative to the baseline strain. In another example, a separate impact score is calculated for every day, practice, game, event, etc. Any trends in the user's impact scores can be identified, which in turn can inform the user of any relevant treatments or behavior modifications. For example, if an impact score is calculated for a football player during each game, and the impact scores show that the user is trending toward experiencing more strain, the user may decide to adjust their recovery practices later in the season.

When the impact score is calculated relative to some baseline impact score (such as the user's baseline impact score or the impact score of a similar population), a variety of different statistical techniques can be used to quantify the user's impact score, such as confidence intervals, deviations, etc. These statistical techniques can also be used to identify which populations the user may be an outlier in, for example by holding a variety of characteristics (e.g., age, sex, height, weight, sport, etc.) constant.

In some implementations, the impact score can be based at least in part on various models of the user's head, brain, and/or other body parts. For example, finite element modeling can be used to model the user's brain tissue, which can in turn be used to characterize impacts the user has received and calculate the impact score. A variety of other techniques can also be used to provide data for the impact score, including machine learning and artificial intelligence.

In some implementations, the impact score can provide an indication of the user's readiness to return to their sport. For example, the impact score can indicate to the user that while the user may not have any actual neurological damage, due to previous impacts, the user may be more susceptible to future neurological damage if they participate in a practice or a game now, instead of resting further. In some implementations, the impact score provides an indication of the potential damage that can be caused by future impacts. Generally, future impacts can result in greater damage to a user if the user has suffered from repeated impacts in the past. The impact score can thus indicate to the user that impacts experienced in an upcoming practice or game could potentially cause more damage than would otherwise be expected due to the user's past experiences.

In some implementations, the impact score quantifies the cumulative number of impacts that the user experiences over a time period. In these implementations, the impact score could be the raw number of impacts experienced. Thus, if the user experiences 15 impacts during a time period (which may include an activity such as a football game), the user's impact score for the time period will be 15. In some implementations, the impact score quantifies an average number of impacts experienced by the user over a certain time period. In these implementations, the impact score could be the rolling average number of impacts experienced every sub-period across a larger time period. For example, if the user has experienced an average of 4 impacts per hour (the sub-period) across the past day, week, month, etc. (the larger time period), the user's impact score will be 4.

In some implementations, the impact score quantifies the amount of force that the user experiences due to impacts, which could be linear force, rotational force, or any combination of the two. In these implementations, the impact score could be the total amount of force experienced by the user during a time period, the average amount of force per impact experienced by the user during the time period, the rolling average amount of force experienced per time period, or other quantifications of force. In other implementations, the impact score quantifies the amount of movement of the user and/or the user's head due to impacts. The amount of movement could be linear, rotation, or a combination of both, and could by the total amount of movement experienced by the user during a time period, the average amount of movement per impact experienced during the time period, the rolling average amount of movement experienced per time period, or other quantifications of movement.

The impact score can also quantify impacts and/or force weighted by a variety of factors. For example, in some cases it may be determined that impacts above a certain threshold amount of force (linear and/or rotational) are more detrimental to a user's health than impacts below the threshold. In these cases, when counting the total number of impacts and/or any average number of impacts, impacts that satisfy the threshold amount of force (e.g., impacts that impart at least the threshold amount of force or more than the threshold amount of force) may be weighted more heavily. Thus, if the impact score quantifies a weighted number of impacts and impacts satisfying the threshold count twice as much as impacts that do not satisfy the threshold, a user that experiences 3 impacts that satisfy the threshold and 3 impacts that do not, the impact score would be 9.

The number of impacts or the imparted force could also be weighted in different ways. For example, impacts imparting substantially or wholly linear force can be weighted differently than impacts imparting substantially or wholly rotational force. In another example, impacts can be weighted based on prior impacts. An impact experienced by a user with a low impact score and/or a user who has not experienced a significant number impacts during the time period that is being measured may be weighted less heavily than an impact experienced by a user with a high impact score and/or a user who has experienced a significant number of impacts during the time period. Thus, repeated impacts within a given time period can begin to contribute more heavily to the user's impact score. In a further example, impacts that result in the user and/or the user's head moving a longer distance (linear and/or rotational) can be weighted heavier than impacts resulting in the user and/or the user's head moving a shorter distance (linear and/or rotational).

Finally, at step 206 of the method 200, the calculated impact score can be communicated to the user. The impact score can be calculated in a variety of different ways. In some implementations, the impact score can be displayed on a screen of the smart device 50, which may be executing a mobile application configured to display the impact score. In other implementations, the impact score can be emailed or texted to the user. In additional implementations, the impact score can be audibly communicated to the user, for example via a speaker of the smart device 50. In further implementations, the impact score can be communicated to the user through the use of visual signals (e.g., LEDs on the mouth guard 1), vibratory signals (e.g., using a buzzer of the mouth guard 1), haptic signals, or other techniques.

Method 200 can be implemented using any combination of devices, systems, etc. For example, in some cases, method 200 is using the mouth guard 1. In these implementations, the impact data is generated by the sensor on the mouth guard 1 (which can include the high-G accelerometer 30, the magnetometer 31, and/or the low-G accelerometer/gyroscope 32), and the impact score is communicated to the user using components on the mouth guard, such as the LED 10 and/or the notification component 36. By using the mouth guard 1 to generate the impact data, a more accurate determination of the impact score can be obtained (for example, as compared to watching the user during an activity or reviewing video of the activity). And by using the mouth guard 1 to notify the user of their impact score (for example if their impact score reaches a dangerous level), the user can be quickly notified if they need to stop participating in the activity. In some cases, the impact data is analyzed by the processor 28 of the mouth guard 1. In these implementations, the impact data does not need to be communicated to a different system or device, and thus the user's impact score can be quickly determined and communicated to them.

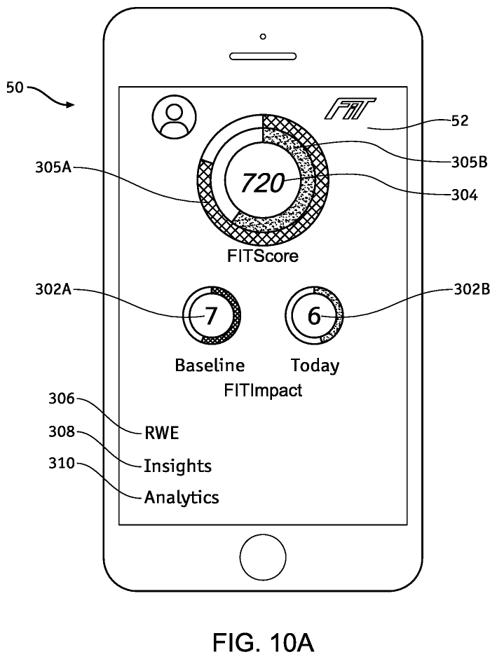

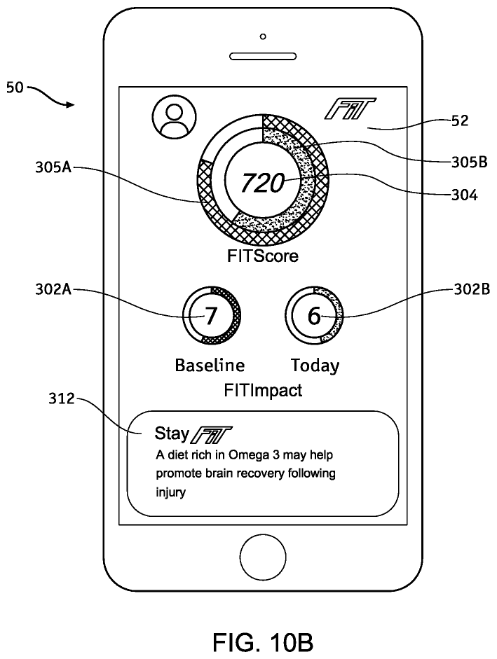

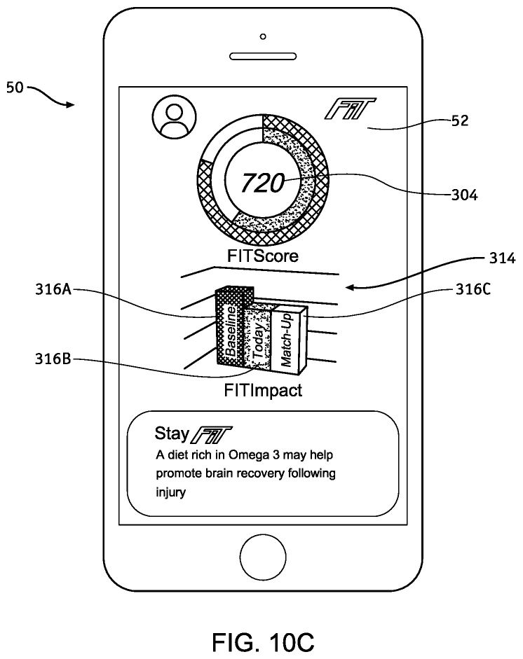

In additional or alternative implementations, a smart device (such as smart device 50 of FIG. 8) can be used to implement at least a portion of method 200. FIG. 10A illustrates the smart device 50 that executes a mobile application that can be used to calculate and/or monitor the user's impact score. The smart device includes a display 52 used to display a variety of different information. In FIG. 10A, the display 52 is displaying a first screen of the mobile application. As shown, the first screen includes an average impact count indicator 302A, a current impact count indicator 302B, and an impact score indicator 304. The average impact count indicator 302A can display a rolling average of the number of impacts experienced by the user over a certain time period, such as a day, week, month, etc. The current impact count indicator 302B indicates the number of impacts experienced by the user for only the current time period being measured. The current impact count indicator 302B can thus be a daily impact count that is reset at the end of each day, a weekly impact score that is reset at the end of each week, etc.

By providing both the average impact count indicator 302A and the current impact count indicator 302B, a user can quickly compare the number of impacts experienced during the current time period being measured with an average number of impacts. Generally, the time period over which the current impact count indicator 302B tallies up impacts will be equal to the time period for which the average impact count indicator 302A applies. Thus, if the average impact count indicator 302A indicates to the user an average number of impacts experienced per day, the current impact count indicator 302B will indicate to the user the number of impacts experienced during the current day. The impact score indicator 304 provides an indication to the user of the user's impact score over a time period. In some implementations, the impact score indicator 304 shows the user's impact score over the same time period as the average impact count indicator 302A and the current impact count indicator 302B.

The impact score indicator 304 can show the impact score in a variety of different manners. In the illustrated implementation, the impact score indicator 304 shows the impact score as a numerical value within a given range. In some implementations, a lower score indicates less strain and a higher score indicates more strain. In some implementations, a lower score indicates more strain and a higher score indicates less strain. The impact score indicator 304 could also show the impact score graphically, for example with a continually updated plot or gauge.

As shown in FIG. 10A, the first screen shown on the display 52 can also include average impact count gauge 305A and a current impact count gauge 305B. The average impact count gauge 305A and the current impact count gauge 305B are both circular gauges formed concentrically with the impact score indicator 304. The gauges 305A and 305B are initially unfilled, and can be filled in with a solid color as the average impact count and the current impact count increase. Thus, the average impact count and the current impact count can be communicated to the user in a variety of different ways on the display 52, including numerically and graphically.

The information presented by the average impact count indicator 302A, the current impact count indicator 302B, the impact score indicator 304, the average impact gauge 305A, and the current impact count gauge 305B can be updated in a variety of different ways. In some implementations, this information is continually updated each successive time period, which could be every minute, every hour, etc. In other implementations, this information is only updated when the application is initially started on the smart device 50, and/or when the user specifically causes the information to be updated, for example by selecting a user-selectable icon that can be displayed on the display 52.

In some implementations, the first screen shown on the display 52 can also show indicators that represent average impact scores and/or average impact counts for users in the same population as the user. For example, the first screen could show an average impact count indicator that shows a daily rolling average impact count for a user of the same age, sex, and sport as the user in question, and/or an average impact score indicator that shows a daily rolling average impact score for a user of the same age, sex, and sport as the user in question. These indicators can allow the user to easily compare their own impact counts and impact scores against similarly-situated users.

In some implementations, the first screen shown on the display 52 can also include an RWE indicator 306, an Insights indicator 308, and an Analytics indicator 310. Each of these indicators can be user-selectable to take the user to a different screen. Selecting the RWE indicator 306 can cause the display 52 to display a screen showing the user's risk weighted exposure score for a certain time period (such as a day). The user's risk weighted exposure is a sum of the concussion probability risk for each impact that the user experiences during a certain time period.

FIG. 10B shows a second screen that can be displayed on the display 52 of the smart device 50 when the user selects the Insights indicator 308. As shown, the display 52 when displaying the second screen can include the average impact count indicator 302A, the current impact count indicator 302B, the impact score indicator 304, the average impact gauge 305A, and the current impact gauge 305B. However, the display 52 also shows a notification indicator 312 that can provide the user with notifications related to the user's impact score. In some implementations, the notifications take the form of text displayed within the notification indicator 312. In some implementations, the notifications provided by the notification indicator 312 can alert the user to negative milestones related to the user's impact score, for example (i) a certain number of consecutive days with an impact score over a threshold value, (ii) a certain number of consecutive days with at least a threshold number of impacts, (iii) a certain number of consecutive days with at least one impact that imparts a force equal to at least a threshold force. However, the notification indicator 312 can notify the user of positive milestones as well.

In other implementations, the notification indicator 312 can provide the user with recommended actions to take to improve the user's impact score in the future. These recommended actions may include increasing the user's hydration, getting more sleep, performing guided breathing and/or meditation, performing moderate outdoor activity (such as walking or biking), decreasing the user's screen time, making dietary changes (for example increasing the user's intake of omega-3 fatty acids), undergoing cold therapy (e.g., an ice bath or cryotherapy treatment), etc. The recommended actions may aid in reducing the risk of exacerbating any injuries or potential injuries by continuing to practice or play in games. In some implementations, techniques such as machine learning and artificial intelligence can be used to determine one or more recommended actions. In some implementations, the recommended actions may be determined relative to previously-recommended actions. For example, if it was previously recommended to the user to engage in some type of therapeutic activity (e.g., moderate outdoor exercise with no screen time), the currently-recommended action can be based on whether the user's impact score has improved or worsened since the previously-recommended action was undertaken. Thus, the currently-recommended action can include increasing the level of therapeutic activity, maintaining the level of therapeutic activity, decreasing the level of therapeutic activity, or ending the therapeutic activity.

In some implementations, the notifications can also provide the user updated information after the user takes one or more recommended actions. For example, the user can be notified if their impact score decreases after increasing hydration for a week and getting more sleep during the week. In these implementations, a score that characterizes the user's recovery or rebound can generated and transmitted to the user. This allows the user to have a tangible indication of how their actions have improved their impact score, and whether the user is able to safely return to their sport. This score could also indicate to the user how much quicker they were able to return to their sport by following the recommended actions, which can incentivize the user to follow the recommended actions in the future. In some implementations, the score characterizing the user's recovery could be the impact score itself. As the user takes one or more recommended actions, the impact score will decrease/improve, indicating to the user the degree of recovery. In other implementations, the score characterizing the user's recovery can be a separate score from the impact score.

In some implementations, the mobile application can proactively notify the user, instead of only notify the user when the user selects the Insights indicator 308. For example, if the user has back-to-back days with an elevated impact score, or if the user has an impact score that is a certain amount greater than the previous day's impact score, the mobile application can notify the user of this occurrence. The mobile application can also proactively notify based on impact counts, instead of just the impact score. For example, a user could experience a large number of impacts within a short time period, but still have a generally low impact score if the impacts are small in magnitude. While the repeated impacts may not be enough on their own to warrant concern, the user may wish to be notified about the repeated impacts so that they can take precautions in the future, so as to avoid more impacts.

FIG. 10C shows a third screen that can be displayed on the display 52 of the smart device 50 when the user selects the Analytics indicator 310. As shown, the display 52 when displaying the third screen can show the impact score indicator 304, along with a plot 314 that provides information related to the current impact score. The information provided by plot 314 aids in explaining to the user whether their current impact score is at, above, or below some standard (which could be a rolling average impact score, a baseline impact score for the user's population, etc.). In the illustrated implementation, the plot 314 includes a data marker 316A indicating the user's average impact count for the relevant time period, a data marker 316B indicating the user's current impact count for the relevant time period, and a data marker 316C that can provide a comparison to other users in the same or similar population. For example, the data marker 316C can indicate the average daily number of impacts experienced by a user of the same age, sex, and sport played as the user in question.

In some implementations, the display 52 can show historical trends for the various types of information as well. The history of the number of impacts, the impact score, the risk weighted exposure, the concussion probability index, and any other relevant information can be shown to the user, which allows the user to easily identify any trends. Additional information can also be shown alongside the historical trends, so that the user can correlate any trends with potential causes, such as diet changes, exercise changes, certain events, etc.

In some implementations, the mobile application being executed on the smart device 50 can allow the user to compare their own data to averages in similar populations. In some implementations, the display 52 can display one or more user-selectable icons that allow the user to select applicable values of various different categories, and then see average impact scores for that population. For example, a 15-year-old female who plays soccer can select those filters, and then compare her impact score to a national average impact score for her population.

Thus, the smart device 50 can be used to implement at least a portion of method 200. For example, the impact data can be communicated from the mouth guard 1 to the smart device 50 using a transmitter (such as low energy Bluetooth transceiver 29). One or more processors on the smart device 50 can analyze the impact data to determine the impact score, and notify the user of the impact score using the display 52. In some cases, the smart device 50 can communicate the impact score back to the mouth guard 1, which can then notify the user. In further implementations, the impact data can be transferred to the cloud, and the smart device 50 can access the impact data from the cloud, or can access a determined impact score from the cloud.

In some cases, method 200 can be implemented using one or more processors, and one or more memory devices having stored thereon machine-executable instructions. The processors and the one or more memory devices, can be located in any one or more locations (such as a mouth guard, a smart device, the cloud, etc.). When the one or more processors execute the machine-executable instructions, method 200 (or variations thereof) is implemented. In some cases, the one or more processors and/or the one or more memory devices can be included as part of a control system, which can be a single device or system (e.g., a smart device), or multiple devices or systems (e.g., a mouth guard, a smart device, the cloud, or any combination). In some cases, instructions for implementing method 200 and/or variations thereof are stored on a computer program product. When executed by a computer, method 200 and/or variations thereof are implemented. The computer program product can be a non-transitory computer readable medium.

These embodiments and obvious variations thereof is contemplated as falling within the spirit and scope of the claimed invention, which is set forth in the following claims. Moreover, the present concepts expressly include any and all combinations and subcombinations of the preceding elements and aspects.

We claim:

1. A method for characterizing impacts experienced by a user, the method comprising:
receiving a plurality of types of data, the plurality of types of data including impact data associated with one or more impacts experienced by the user, and demographic data associated with the user;
determining, based at least in part on the impact data and the demographic data, one or more impact scores for the user, the one or more impact scores characterizing an effect on the user from the one or more impacts; and
communicating the one or more impact scores to the user.

2. The method of claim 1, wherein the impact data includes (i) data related to a linear velocity of the user associated with the one or more impacts, (ii) data related to a rotational velocity of the user associated with the one or more impacts, (iii) data related to a linear acceleration of the user associated with the one or more impacts, (iv) data related to a rotational acceleration of the user associated with the one or more impacts, (v) data related to an amount of linear force imparted to the user by the one or more impacts, (vi) data related to an amount of rotational force imparted to the user by the one or more impacts, (vii) data related to a duration of the one or more impacts experienced by the user, (vii) data related to a time between successive impacts of the one or more impacts, (ix) data related to a direction of the one or more impacts impact relative to a baseline direction, or (x) any combination thereof

3. The method of claim 1, wherein the demographic data includes the user's age, the user's sex, the user's height, the user's weight, the user's medical history, a sport that the user plays, a position in the sport that the user plays, or any combination thereof.

4. The method of claim 1, wherein the plurality of types of data further includes environmental data associated with an area where the user is located, the environmental data including an ambient temperature of the area, an elevation of the area, a precipitation level of the area, a humidity of the area, an air quality level of the area, or any combination thereof.

5. The method of claim 1, wherein the one or more impact scores includes a daily impact score, a weekly impact score, a monthly impact score, a rolling average impact score, or any combination thereof.

6. The method of claim 5, wherein the daily impact score is based on impacts received during a current day and during at least one day prior to the current day.

7. The method of claim 5, wherein the rolling average impact score is a daily rolling average impact score.

8. The method of claim 7, wherein the daily rolling average impact score is an average daily impact score across each of seven days prior to a current day.

9. The method of claim 1, further comprising communicating to the user one or more recommended actions to improve at least one of the one or more impact scores.

10. The method of claim 9, wherein the one or more recommended actions includes increasing hydration, increasing an amount of sleep, participating in moderate outdoor activity, reducing an amount of time spent by the user viewing an electronic display, a diet change, abstaining from a sport played by the user for a period of time, or any combination thereof.

11. The method of claim 1, wherein the one or more impact scores are indicative of a risk of concussion from the one or more impacts, a risk of neurological damage from the one or more impacts, an amount of neurological damage caused from the one or more impacts, a risk of non-neurological damage from the one or more impacts, an amount of non-neurological damage caused by the one or more impacts, or any combination thereof.

12. The method of claim 1, wherein the impact data is generated using one or more sensors located in a mouth guard worn by the user.

13. The method of claim 12, wherein the one or more sensors include an accelerometer, a gyroscope, a magnetometer, or any combination thereof.

14. The method of claim 12, wherein the mouth guard includes one or more light emitting diodes, one or more notification components, or both, and wherein the one or more impact scores are communicated to the user using the one or more light emitting diodes, the one or more notification components, or both.

15. The method of claim 14, wherein the one or more notification components include a buzzer, a speaker, a piezoelectric element, a magnetic element, or any combination thereof.

16. A system for characterizing impacts experienced by a user, the system comprising:
one or more memory devices having stored thereon machine-readable instructions; and
one or more processors configured to execute the machine-readable instructions to:
receive a plurality of types of data, the plurality of types of data including impact data associated with one or more impacts experienced by the user, and demographic data associated with the user;
determine, based at least in part on the impact data and the demographic data, one or more impact scores for the user, the one or more impact scores characterizing an effect on the user from the one or more impacts; and
communicate the one or more impact scores to the user.

17. The system of claim 16, wherein the one or more processors are located in a mouth guard worn by the user, in a smart device, or both.

18. The system of claim 17, wherein the impact data is generated using one or more sensors located in the mouth guard.

19. The system of claim 18, wherein the one or more impact scores are determined using one or more processors of the smart device.

20. The system of claim 18, wherein the one or more impact scores are determined using one or more processors of the mouth guard.
